(19) United States
(12) Patent Application Publication
(10) Pub. No.: US 2008/0108698 A1
(43) Pub. Date: May 8, 2008

(54) Nutrient Supplement and Use of the Same

(75) Inventors: Pentti Hietala, Vantaa (FI); Tuomo Karila, Ostersundom (FI); Timo Seppala, Helsinki (FI); Karim Tahtivuori, Helsinki (FI)

Correspondence Address: Seyfarth Shaw, 131 S. Dearborn, Suite 2400, Chicago, IL, 60603, US

(73) Assignee: OY ELMOMED LTD, OSTERSUNDOM (FI)

(21) Appl. No.: 11/666,028

(22) PCT Filed: Oct. 20, 2005
(86) PCT No.: PCT/FI05/50365

§ 371 Date: Jul. 30, 2007

(30) Foreign Application Priority Data
Oct. 21, 2004 (FI) 20045395

(51) Int. Cl. A61K 31/19; A61K 31/22; A61K 31/16; A61P 21/00

(52) U.S. Cl. 514/546; 514/557; 514/613

(57) ABSTRACT

The present invention generally relates to enhancement and recovery of muscle performance in a state of stress induced by physical exercise, disease or trauma. More particularly the invention relates to means for improved muscle performance and for providing more efficient muscle recovery after physical or traumatic stress. The present invention also relates to means for increasing body mass, including muscle mass, and especially lean body mass. Specifically, the invention relates to a nutrient supplement and the use thereof for enhanced recovery and/or performance of the muscles. The invention further relates to a method for improving muscle performance in and muscle recovery from a state of stress induced by physical exercise, disease or trauma.

### FIELD OF THE INVENTION

The present invention generally relates to enhancement and recovery of muscle performance in a state of stress induced by physical exercise, disease or trauma. More particularly the invention relates to means for improved muscle performance and for providing more efficient muscle recovery after physical or traumatic stress. The present invention also relates to means for increasing body mass, including muscle mass, and especially lean body mass. Specifically, the invention relates to a nutrient supplement and the use thereof for enhanced recovery and/or performance of the muscles. The invention further relates to a method for improving muscle performance in and muscle recovery from a state of stress induced by physical exercise, disease or trauma.

### BACKGROUND OF THE INVENTION

Delayed-onset muscle soreness is described as post exercise muscle soreness. It is the sensation of muscular discomfort and pain during active contractions, which occur in a delayed fashion after strenuous exercise. The soreness and accompanying muscle damage are more pronounced, if the exercise performed is new to the individual. Individuals with delayed-onset muscle soreness experience painful, tender, and swollen muscles with reduced range of motion of adjacent joints, especially after unaccustomed exercise. In addition to muscle tenderness with palpation, prolonged strength loss, a reduced range of motion and elevated levels of serum creatine kinase are observed. These symptoms develop during the first 24 to 48 hours and disappear within 2 to 7 days. Delayed-onset muscle soreness symptoms are particularly associated with the eccentric exercise, i.e. a type of exercise where an activated muscle is forced to elongate while producing tension. [Barlas, P., et al., Arch Phys Med Rehabil 2000; 81(7): 966-972, Lieber, R. L. and Friden, J., J Am Acad Orthop Surg 2002; 10(1): 67-73].

Muscle pain after unaccustomed exercise is believed to result from repetitive active lengthening of skeletal muscle. Especially, eccentric resistant training performed with weights results in muscle cytoskeletal breakdown, inflammation, and remodelling (Lieber, R. L. and Friden, J., supra). The pathophysiology of delayed-onset muscle soreness remains still undetermined, but it has been reported that after strenuous exercise muscle cell damage and inflammatory cells are observed in damaged muscle (Barlas, P., et al., supra; Lieber, R. L. and Friden, J., supra). Muscle damage after strenuous eccentric exercise is initialized by proteolytic and lipolytic systems (Barlas, P., et al., supra).

During and after strenuous resistance exercise both muscle protein synthesis and breakdown are increased [Tipton, K. D. and Wolfe, R. R., Int J Sport Nutr Exerc Metab 2001; 11(1): 109-132; Sheffield-Moore, M., et al., Am J Physiol Endocrinol Metab 2004 Sep; 287(3): E513-22]. Particularly, the amount of myofibrillar protein is increased in skeletal muscle after resistance training.

Generally, during all sort of exercise the total protein balance is negative due to increased protein breakdown [Rennie, M. J., et al., Clin Sci (Lond) 1981; 61(5): 627-639]. Recently, Pitkänen et al. [Med Sci Sports Exerc 2003; 35(5): 784-792] reported that the resistance training induced protein breakdown continues also after a bout of exercise. Generally, resistance exercise improves muscle protein balance, but in the absence of food intake, the protein balance remains negative. The response of muscle protein metabolism to a resistance exercise bout lasts for 24-48 hours (Tipton, K. D. and Wolfe, R. R., supra).

People accept muscle soreness as temporary discomfort. However, top athletes prefer to overcome these injuries and be restored to normal function with a minimal disruption to training programs or work output. Standard treatments for muscle pain are rest, ice, compression, elevation and then mobilizing the particular tight tissues until normality is maintained. Also treatments, such as massage or stretching, are employed. These treatments relieve local symptoms, but the mechanical treatment of muscle pain is not always enough. For instance, ice massage reduces the appearance of creatine kinase, but it has no other effect on signs and symptoms associated with the exercise-induced muscle [Howatson G. and Van Someren K. A., J Sports Med Phys Fitness. 2003 Dec; 43(4): 500-505].

As mentioned above, inflammatory cells are observed in damaged muscle. However, since delayed-onset muscle soreness symptoms are not totally due to the inflammatory process, an anti-inflammatory medication does not prevent from isometric strength loss, soreness, tenderness, and decreased muscular function [Pizza, F. X., et al., Int J Sports Med 1999; 20(2): 98-102]. Neither ibuprofen nor paracetamol reduced eccentric resistant training induced muscle soreness [Trappe, T. A., et al., Am J Physiol Endocrinol Metab 2002; 282(3): E551-E556]. Similarly, in a study by Barlas, P., et al. (supra) it was found that neither aspirin, paracetamol nor codeine had a beneficial effect on delayed-onset muscle soreness induced by eccentric exercise during an 11-day study period with 60 study subjects.

A variety of means and methods have been proposed for optimal muscle performance. For example, nutrition is the primary determinant of the outcome of the critical short-term muscle recovery process. The athletes, who pay attention to their nutrition will recover faster and more fully after workouts and therefore perform better in subsequent workouts and become better conditioned.

In many fields of sports, which require physical strength, an increase in lean body mass without the ordinarily attendant increase in fat mass is preferable. For this purpose and for better recovery after exercise, a variety of nutritional supplements are commercially available. Food supplements are typically designed to compensate for reduced levels of nutrients in the diet. In particular, in the field of sports and physical exercise, natural food supplements, which specifically improve athletic ability, are increasingly important, for example, supplements that promote or enhance physical performance. The most common supplements currently used are mixtures of creatine, protein powder, amino acids, vitamins, zinc, copper, and magnesium. In fact, many of these dietary supplements are often promoted as a safe alternative to anabolic steroids, androgen prohormones, growth hormone or other ergogenic substances that receive media attention and whose use is usually to some extend banned in many countries and certainly more controversial.

Despite many mechanical methods for physical recovery and the progress in the knowledge of nutrition, better and simpler ways and means for both fast recovery and increased performance of the muscles are needed. There is a demand for a safe and healthy nutritional supplement having anabolic effects without any side effects. The present invention meets this demand.

### BRIEF DESCRIPTION OF THE INVENTION

An object of the present invention is to provide novel means for the enhancement of performance and recovery of the muscles in a state of stress induced by physical exercise, disease or trauma.

Another object of the present invention is to provide novel means for protecting the muscle cells from breakdown in a state of stress induced by physical exercise, disease or trauma.

Yet another object of the present invention is to provide novel means to balance muscle protein metabolisms after resistance exercise.

Yet another object of the present invention is to provide novel means for enhanced performance and/or recovery of the muscles involved in strenuous physical exercise.

Yet another object of the invention is to provide novel means to enhance muscle performance and to increase body mass, including muscle mass, and especially lean body mass without adverse side effects.

Still a further object of the invention is to provide novel means for enhanced performance and/or recovery of the muscles involved in strenuous physical exercise, which are suitable for both athletes and fitness trainers.

Still a further object of the invention is to provide means for enhanced performance and/or recovery of the muscles involved in strenuous physical exercise, which are easy and safe to use and allow both better recovery and increased body mass.

Still a further object of the present invention is to provide novel means for the treatment and prevention of delayed onset muscle soreness symptoms.

Still a further object of the invention is to provide novel means to enhance performance and recovery of the muscles and/or to increase muscle mass after a long-term immobility irrespective of the cause of immobilization.

It was surprisingly found that the objects of the present invention are achieved by the use of DL-α-hydroxy-isocaproic acid (HICA) or physiologically acceptable ester and amide derivatives and salts thereof as a nutrient supplement.

Accordingly, the present invention relates to the use of DL-α-hydroxy-isocaproic acid (HICA) and physiologically acceptable ester and amide derivatives and salts thereof as a nutrient supplement for enhancement of performance and recovery of the muscles in a state of stress induced by physical exercise, disease or trauma. In a preferred embodiment of the invention DL-α-hydroxy-isocaproic acid (HICA) or a physiologically acceptable salt thereof is used.

The present invention also relates to a nutrient supplement composition comprising DL-α-hydroxy-isocaproic acid (HICA) or a physiologically acceptable ester or amide derivative or salt thereof.

The present invention further relates to a method for improving muscle performance in and muscle recovery from a state of stress induced by physical exercise, disease or trauma, comprising administering an amount of DL-α-hydroxy-isocaproic acid (HICA) or a physiologically acceptable ester or amide derivative or salt thereof, sufficient to enhance the performance and/or recovery of the muscles in a state of stress induced by physical exercise, disease or trauma, to a subject in need thereof.

### DETAILED DESCRIPTION OF THE INVENTION

The present invention is based on a surprising finding that the administration of DL-α-hydroxy-isocaproic acid (HICA) to athletes decreases muscle pain after extensive training and also increases lean body mass, including muscle mass, without increasing body fat.

HICA (DL-α-hydroxy-isocaproic acid; synonyms: DL-2-hydroxy-4-methylvaleric acid, L-leucic acid) is a normally occurring metabolite in mammalian organisms including humans. It is the main end product in the metabolism of branched-chain amino acid leucine. It is non-toxic having LD₅₀ (iv. in mice, Na-salt) of 650 mg/kg. HICA is commercially available (e.g. Aldrich) as colorless crystals with sweet and sour taste and is soluble in water and alcohols.

U.S. Pat. No. 6,203,835 discloses the use of α-hydroxy-isocaproic acid as an antimicrobial component in animal feed for promoting animal growth and improving feed utilization efficiency. It is speculated that the obtained effects are due to antimicrobial properties of α-hydroxy-isocaproic acid. The growth promoting effect is achieved when α-hydroxy-isocaproic acid is administered in combination with another branched carbon chain hydroxy acid.

WO97/00676 discloses the use of α-hydroxy-isocaproic acid in the manufacture of a preparation useful for antimicrobial and/or proteinase activity-inhibiting efficacy. The use is based on the inhibitory and bactericidal efficacy of α-hydroxy-isocaproic acid on microorganisms and proteinases, particularly on the inhibition of matrix metalloproteinases and serine proteinases.

It was unexpectedly found that the use of HICA or physiologically acceptable ester or amide derivatives or salts thereof as a nutrient supplement 35 enhances performance and/or recovery of the muscles in a state of stress induced by physical exercise, such as long-term strenuous physical exercise, and in states involving muscle cell loss or breakdown, such as those following surgical operations, ruptures or other disorders, which may cause muscle breakdown.

For the present purposes the expressions “enhanced performance of the muscles” or “enhanced muscle performance” mean that the irritability, conductivity, adaptivity and contractility of the muscles are better with the use of HICA than without the use of HICA. During an intensive training period the athletes experience improved muscle capacity when using HICA. For the present purposes the expressions “enhanced recovery of the muscles” or “enhanced muscle recovery” mean that the muscles are restored to normal level of function faster with the use of HICA than without the use of HICA. Normally the symptoms of delayed onset muscle soreness develop during the first 24 to 48 hours. After the intake of HICA the subjective symptoms are significantly reduced or even disappear, and also shorter recovery periods and less recovery therapy are needed. The use of HICA additionally enhances power performance. For the present purpose “enhanced power performance” means that the ability of muscle to contract at a force and speed, which maximizes power, is better with the use of HICA than without the use of HICA.

For the present purposes “strenuous exercise” refers to the activity of exerting muscles in various ways to keep fit, which activity is characterized by or performed with much energy or force. For the present purposes “state of stress induced by physical exercise, disease or trauma” of the muscle means that the muscle is in a metabolic state where the total protein balance is negative due to increased protein breakdown. In trained muscle this leads to symptoms of aching, tender, and swollen muscles with reduced range of motion and rigidity, and prolonged strength loss. In trauma this leads often to atrophy and immobilization of the muscle. Diseases that induce state of stress in muscles include all diseases or disorders involving muscle cell damage or muscle loss, such as catabolic conditions and muscular dystrophy.

The effect of HICA is observed in any physical state, which involves muscle stress. Such physical states include states, where the muscle is under physical muscle work, for instance during strenuous exercise performed by an athlete or during an unaccustomed bout of exercise performed by a fitness trainer; states, where the muscle is recovering from physical work after strenuous exercise; states, where the muscles are immobilized for prolonged period of times due to, for instance, a surgical operation, a bone fracture, poor general condition, or a disease, and similar states. HICA exerts thus an anti-catabolic function, this function is especially pronounced during and/or after the strenuous exercise.

The use of HICA reduces the sensation of muscular discomfort and pain during active contractions that occurs in delayed fashion after strenuous exercise. These symptoms develop during the first 24 to 48 hours and disappear within 2 to 7 days. The pathophysiology of delayed-onset muscle soreness has not been elucidated, but it is believed that it at least in part involves muscle cell damage. With the intake of HICA the symptoms of rigidity, pain, stiffness and aches of the muscles are relieved and even abolished after both strenuous resistance and/or endurance training and also after a bout of strenuous exercise. This affords the athletes and fitness trainers to continue their exercise with full intensity sooner.

One advantage of the use of HICA is that it minimizes the loss of muscle mass and even increases lean body mass without any changes in the bone or fat tissue masses. For athletes, the increase in lean body mass is desired, because the energy required for the muscle performance is produced faster by the muscles compared to fat tissue. The use of HICA also enhances muscle performance and increases muscle mass without adverse side effects.

The fact that HICA reduces the muscle cell damage caused by strenuous exercise also suggests its usefulness in the adjuvant therapy of diseases or disorders involving muscle cell damage or muscle loss, such as catabolic conditions and muscular dystrophy and in therapy of muscle damage and muscle loss after burns, surgery, trauma, long-term immobilization and like.

One important feature of the use of HICA is that HICA exerts its effect when administered alone. A typical effective dosage of HICA can be around or less than 20 mg/kg/day of HICA. This means that the daily dose is in the range of a few grams per day in comparison to the amounts of about 100 to 300 grams per day of the most of conventional nutrient supplements. The range of the HICA dosage is 5-100 mg/kg/day, preferably from 10-40 mg/kg/day, and most preferably 15-20 mg/kg/day. However, the dosage may be higher or lower than these, since naturally the suitable dose depends on the individual, the nature and intensity of training (endurance training vs. a bout of training), the personal diet, age, gender and similar factors.

An additional advantageous feature of HICA is that it simultaneously induces fast recovery, enhanced power performance and increased lean body mass. Accordingly, the use of several different nutrient supplements is unnecessary. HICA does not have any energy content with the given dosage and thus does not disturb energy balance/diet.

For enhancement of performance and recovery of the muscles, a suitable dose of HICA or a physiologically acceptable ester or amide derivative or salt thereof is taken after each training session. However, for periodic or long-term use the timing of the intake is not critical as long as the blood levels of HICA remain at levels sufficient for HICA to exert its function. For athletes, these blood levels are achieved by administration, for example, two to four times per day. Generally, it is suggested that HICA be taken immediately after the training period, preferably within 1 to 3 hours after the training session. However, the alleviation of the delayed onset muscle soreness symptoms may be achieved by the intake of HICA even after up to 24 hours after the training session.

When HICA or physiologically acceptable ester or amide derivatives or salts thereof are administered to subjects at a risk of or having muscle mass loss due to immobilization or any other condition mentioned above, the administration on continuous basis for as long as the state of immobilization continues is preferred. Thus, for example, for a subject having a bone fraction in leg should take a HICA supplement for at least 4 to 8 weeks.

The nutrient supplement of the invention comprising of HICA or physiologically acceptable ester or amide derivatives or salts thereof is administered by any suitable route, such as orally, intramuscularily or intravenously. The oral route is preferred. A suitable dosage form for oral administration is a solid dosage form, such as a tablet, capsule, granule, microgranule or powder, or a liquid dosage form, such as a solution, suspension or injectable solution. One preferred solid dosage form for oral administration is a compressed or coated tablet. Other preferred solid forms for oral administration are granules and powders, which can upon use be dissolved in a suitable liquid such as water, juice, milk, and like. Alternatively the nutrient supplement of the invention can be in a form of drink mixes, bars, soft gels and like. For the intramuscular or intravenous administration HICA is dissolved in a solvent suitable for injection, such as physiological saline.

The nutrient supplement of the present invention preferably contains only HICA or physiologically acceptable ester or amide derivatives or salts thereof. Suitable salts include physiologically acceptable inorganic salts, such as ammonium, sodium, potassium, calcium, magnesium and similar salts, and physiologically acceptable organic salts. However, it may contain in addition to HICA any other acceptable carriers, excipients and additives, which are necessary for the formulation of the final HICA preparation. Suitable additives include buffers, flavors, aromic agents, sweeteners and like.

In one aspect, the present invention provides a method for improving muscle performance in and muscle recovery from a state of stress induced by physical exercise, disease or trauma. In the method of the invention, HICA or a physiologically acceptable ester or amide derivative or salt thereof is administered in amounts sufficient to enhance the muscle performance and/or recovery of the muscles in a state of stress induced by physical exercise, disease or trauma to a subject in need thereof. These amounts and the subjects are as described above.

According to the present invention HICA is useful for both top athletes and normal fitness trainers. Additionally, it is useful for subjects at a risk of having muscle mass loss due to immobilization of any cause. When HICA was administered to 7 voluntary healthy top athletes, these athletes reported that HICA reduced pain, stiffness and aches after training and caused enhanced power performance without any adverse effects. An additional advantage of the use of HICA was that it increased lean body mass without any changes in bone or fat tissue masses: the mean weight gain during the 42-day treatment was 0.8 kg (see Example 1). The use of HICA as a nutrient supplement can be thus promoted as a safe alternative to conventional nutrient supplements.

The present invention provides an easy and simple way for recovery after physical exercise and increased muscle performance. The use of the nutrient supplement composition of the invention provides enhanced power performance and reduced muscle soreness, increased lean body mass and decreased catabolism in muscle tissue.

The invention will be described in greater detail by means of the following examples. The examples are only intended to illustrate the invention and they are not regarded as restricting the scope of the invention in any way.

### EXAMPLE 1

In order to assess the effects of HICA (α-hydroxy-isocaproic acid) on exercise induced muscle pain and body composition, 0.496 g of HICA (produced in VTT Technical Research Centre of Finland, Helsinki) was given thrice daily after intensive training sessions to 7 healthy volunteers for 42 days in an open study. The volunteers were national top wrestlers, weighing 79.7+/−4.5 kg (mean+/−SD) and aging 26+/−6 years (mean+/−SD). They had at least 10 training sessions a week, each lasting from 1.5 to 2.5 hours.

During 6 weeks preceding the HICA period there were no essential changes in the body weight of the wrestlers. At least for 6 weeks before and during the trial daily diets and the number, intensity, and length of daily training sessions were kept constant.

Before the study the subjects underwent a medical examination. Twenty ml of blood was taken for chemical assays and the body weight and body composition were assessed by dual-energy X-ray absorptiometry (DEXA; LUNAR GE Medical Systems) just before starting the 42-day HICA intake.

The subjects took HICA orally as liquid (62.5 g HICA dissolved in 630 ml water and buffered by NaOH to pH 3.8). The single dose taken three times a day after each training session was 5 ml (containing 0.496 mg of HICA) of the solution mixed with apple juice. On those days they had less than three training session they took extra doses of HICA so that 3 doses were taken each day. The total daily dose was 1488 mg of HICA.

Subjects were asked to report all feelings they would associate with the treatment with HICA, e.g. pain, stiffness or aches in muscles felt during and after the training sessions.

All 7 subjects associated the treatment with HICA with the abolishment of pain, stiffness and aches in muscles felt during and after the training sessions. Subjectively 6 out of 7 subjects reported enhanced power performance after the treatment with HICA.

The results of measurable parameters showed that mean+/−SD weight gain during the treatment period was 0.84+/−1.0 kg (P<0.05, paired t-test) (Tables 1 and 2). According to DEXA measurements bone weight was not changed but total soft tissue mass (total weight−bone weight) was increased significantly (P<0.05, paired t-test) (Table 1). The soft tissue masses of both all extremities (upper and lower, left and right summarized) and trunk were increased significantly (P<0.05 and P<0.001, respectively) during the treatment with HICA (Table 2).

TABLE 1
Mean +/− SD whole body weight, soft tissue weight, and bone
weight (in kilograms) of the subjects before and after the treatment with
HICA.

| | Whole body | | Soft tissue | | | |
|---|---|---|---|---|---|---|
| | weight* | | weight* | | Bone weight^{NS} | |
| Subject | Before | After | Before | After | Before | After |
| Number | HICA | HICA | HICA | HICA | HICA | HICA |
| 1 | 79.5 | 79.1 | 75.5 | 75.0 | 4.0 | 4.1 |
| 2 | 87.7 | 88.1 | 83.3 | 83.7 | 4.4 | 4.4 |
| 3 | 74.4 | 75.2 | 70.5 | 71.2 | 3.9 | 4.0 |
| 4 | 76.5 | 77.6 | 72.9 | 74.0 | 3.6 | 3.6 |
| 5 | 77.4 | 79.3 | 73.4 | 75.4 | 4.0 | 3.9 |
| 6 | 79.2 | 79 | 75.3 | 75.1 | 3.9 | 4.0 |
| 7 | 83.4 | 85.7 | 79.3 | 81.6 | 4.1 | 4.1 |
| Mean | 79.73 | 80.57 | 75.8 | 76.6 | 4.0 | 4.0 |
| SD | 4.50 | 4.60 | 4.3 | 4.4 | 0.2 | 0.2 |

Paired Two Sample t-Test (Before HICA vs After HICA):
*P < 0.05;
^{NS}= nonsignificant.

TABLE 2
Mean +/− SD total weight of soft tissue (in kilograms) in extremities
and trunk of the subjects before and after the treatment with HICA.

| | Extremities* | | Trunk*** | |
|---|---|---|---|---|
| | Before | | Before | |
| Subject number | HICA | After HICA | HICA | After HICA |
| 1 | 35.5 | 35.2 | 40.0 | 39.8 |
| 2 | 39.6 | 39.0 | 43.8 | 44.8 |
| 3 | 32.9 | 33.2 | 37.6 | 38.0 |
| 4 | 35.7 | 35.7 | 37.2 | 38.3 |
| 5 | 34.4 | 35.7 | 39.0 | 39.7 |
| 6 | 35.5 | 34.7 | 39.8 | 40.4 |
| 7 | 36.7 | 38.5 | 42.6 | 43.1 |
| Mean | 35.8 | 36.0 | 40.0 | 40.6 |
| SD | 2.1 | 2.1 | 2.4 | 2.5 |

Paired Two Sample t-Test (Before HICA vs After HICA):
*P < 0.05;
***P < 0.001.

TABLE 3
Blood pressure, heart rate and clinical chemistry before and after
the treatment with HICA (n = 7).

| | | Before | | | | |
|---|---|---|---|---|---|---|
| | | HICA | | After HICA | | |
| Parameter | Unit | Mean | SEM | Mean | SEM | Significance |
| systolic blood | mmHg | 148.6 | 4.2 | 139.7 | 4.8 | * |
| pressure | | | | | | |
| diastolic blood | mmHg | 83.0 | 3.1 | 75.7 | 2.7 | ** |
| pressure | | | | | | |
| heart rate | 1/min | 58.7 | 3.3 | 62.0 | 3.8 | NS |
| fS-Creatinine | mmol/l | 88.7 | 2.9 | 89.2 | 3.6 | NS |
| S-Alanine amino | U/l | 32.3 | 2.2 | 31.6 | 2.7 | NS |
| transferase | | | | | | |
| B-Hematocrite | % | 43.1 | 0.8 | 42.6 | 0.6 | NS |
| B-Hemoglobin | g/l | 153.4 | 3.1 | 149.1 | 3.5 | NS |
| B-Erythrocytes | E12/l | 5.0 | 0.1 | 4.9 | 0.1 | NS |
| B-Leucocytes | E9/l | 5.7 | 0.3 | 5.7 | 0.6 | NS |
| E-MCHC | g/l | 340.9 | 2.2 | 347.9 | 3.5 | NS |
| E-MCH | pg | 30.6 | 0.4 | 30.4 | 0.4 | NS |
| E-MCV | fl | 89.4 | 1.1 | 87.4 | 1.1 | NS |

Paired Two Sample t-Test (Before HICA vs After HICA):
*P < 0.05;
**P < 0.01.

The results suggest that a 42-day treatment with HICA causes increased soft tissue mass, abolishes exercise related muscle pain and stiffness, and enhances subjectively power performance without any adverse effects.

### EXAMPLE 2

A basket ball player (age 36 yr; weight 83.7 kg; BMI 26.8 kg/m²) took after intensive daily training sessions 0.496 g of HICA three times a day for 42 days in an identical design as described in Example 1. The composition of his soft tissue was analyzed in detail. DEXA-results were analyzed by a software discriminating successfully between bone, fat and lean body mass.

According to DEXA-results the volunteer gained 2.65 kg of lean body mass during the treatment by HICA (Table 4). Subjectively he reported the disappearance of all exercise related muscle aches and pains. Laboratory tests, e.g. blood pressure, heart rate or blood analyses showed no changes (data not shown).

This case study suggests that HICA combined with intensive training has a muscle building effect.

TABLE 4
Body composition of a basketball player before and after 42-day
treatment with HICA.

| | Before HICA | After HICA | Change (g) |
|---|---|---|---|
| bone (kg) | 4.1 | 4.1 | +0.0 |
| fat (kg) | 7.8 | 8.1 | +0.3 |
| lean (kg) | | | |
| total | 71.8 | 74.4 | +2.7 |
| extremities | 33.2 | 34.8 | +1.6 |
| trunk | 34.4 | 35.45 | +1.0 |
| other | 4.3 | 4.3 | +0.0 |
| weight (kg) | 83.7 | 86.6 | +3.0 |

1. The use of DL-α-hydroxy-isocaproic acid (HICA) or physiologically acceptable ester or amide derivatives or salts thereof for the manufacture of a nutrient supplement for enhancing performance and recovery of the muscles in a state of stress induced by physical exercise.

2. The use of HICA according to claim 1, wherein said use is before, during and/or after the exercise.

3. The use of HICA according to claim 2, wherein said use is after strenuous physical exercise.

4. The use of HICA according to claim 3, wherein said use reduces pain, stiffness and aches after resistance and endurance training.

5. The use of HICA according to claim 3, wherein said use reduces pain, stiffness and aches after a bout of exercise.

6. The use of HICA according to anyone of claims 1-4, wherein said use enhances muscle performance and increases muscle mass without adverse side effects.

7. The use of HICA according to claim 1 or 2, wherein said use additionally enhances power performance.

8. The use of HICA according to claim 5, wherein said use increases lean body mass.

9. The use of HICA according to any one of the preceding claims, wherein the nutrient supplement is to be administered in a dosage form of 5-100 mg/kg/day.

10. A nutrient supplement composition comprising essentially of α-hydroxy-isocaproic acid (HICA) or a physiologically acceptable ester or amide derivative or salt thereof in orally administerable form.

11. A method for improving muscle performance in and muscle recovery from a state of stress induced by physical exercise, disease or trauma, comprising administering an amount of DL-α-hydroxy-isocaproic acid (HICA) or a physiologically acceptable ester or amide derivative or salt thereof sufficient to enhance the performance and/or recovery of the muscles in a state of stress induced by physical exercise, disease or trauma to a subject in need thereof.
